(19) United States
(12) Plant Patent Application Publication
(10) Pub. No.: US 2004/0083521 P1
(43) Pub. Date: Apr. 29, 2004

(54) Chrysanthemum plant named 'Rio Dark'

(75) Inventor: Keith G. Lintott, Bognor Regis (GB)

Correspondence Address: HOWREY SIMON ARNOLD & WHITE, LLP, BOX 34, 301 RAVENSWOOD AVE., MENLO PARK, CA, 94025, US

(73) Assignee: Cleangro Ltd

(21) Appl. No.: 10/693,363

(22) Filed: Oct. 24, 2003

Related U.S. Application Data
(60) Provisional application No. 60/421,382, filed on Oct. 25, 2002.

(51) Int. Cl. A01H 5/00

(52) U.S. Cl. PLT/297

(57) ABSTRACT

A new variety of Chrysanthemum plant named ‘Rio Dark,’ having a uniform canopy of single dark pink flowers and an upright habit. The new variety has shown good weather resistant characteristics.

This application claims the benefit of U.S. Provisional Application No. 60/421,382, filed Oct. 25, 2002.

### LATIN NAME OF THE GENUS AND SPECIES

Botanical classification: Chrysanthemum morifolium.

### VARIETY DENOMINATION

The new Chrysanthemum variety denomination is ‘Rio Dark.’

### BACKGROUND OF THE INVENTION

The present invention comprises a new and distinct cultivar of Chrysanthemum botanically known as Chrysanthemum morifolium, and referred to by the cultivar name ‘Rio Dark.’

‘Rio Dark,’ identified as 30091-4, originated from a whole plant mutation grown in a controlled planting of the unpatented variety ‘Rio’ in Chichester, West Sussex, United Kingdom. The new variety ‘Rio Dark’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Rio Dark’ is a garden type of Chrysanthemum plant variety having a domed canopy of single dark pink flowers and an upright habit.

### Comparison With Parent

Plants of the new Chrysanthemum variety ‘Rio Dark’ are similar to the parent variety ‘Rio’ in plant habit and growth rate. In side-by-side comparisons in Chichester, West Sussex, United Kingdom, under commercial practice, plants of the new Chrysanthemum variety ‘Rio Dark’ differed from plants of the parent ‘Rio’ in the following characteristic.

1. The new variety ‘Rio Dark’ produces dark pink flowers whereas the parent ‘Rio’ produces light pink flowers.

### Comparison With Other Varieties

Plants of the new Chrysanthemum variety ‘Rio Dark’ are dissimilar to the variety ‘Merriment’ (U.S. Plant Pat. No, 5,285) in plant habit and growth rate. In side-by-side comparisons in Chichester, West Sussex, United Kingdom, under commercial practice, plants of the new Chrysanthemum variety ‘Rio Dark’ differed from ‘Merriment’ in the following characteristics.

1. The new variety ‘Rio Dark’ produces more cupped shaped dark pink flowers whereas ‘Merriment’ produces more flat mid-pink flowers.

2. The new variety ‘Rio Dark’ has a more uniform domed shaped plant habit than the more spreading habit of ‘Merriment.’

### BRIEF DESCRIPTION OF ILLUSTRATION

Typical specimens of the plant and flowers for the new Chrysanthemum variety ‘Rio Dark’ are shown in the accompanying digital photograph. The colors shown are as true as possible within the usual limits of this kind of illustration.

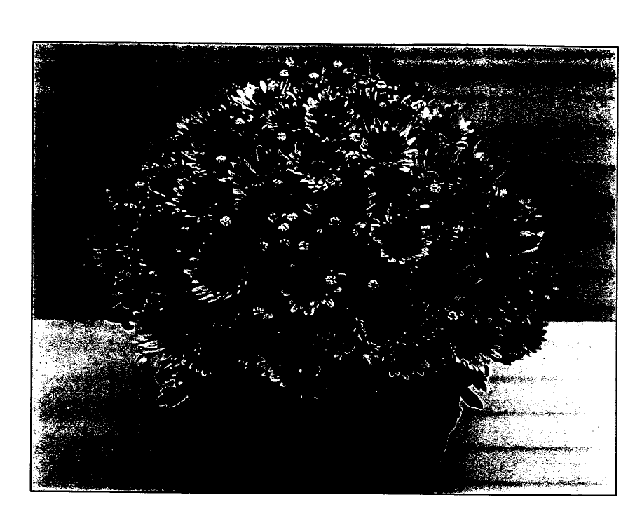

FIG. 1 is a whole plant view of the new variety ‘Rio Dark’ grown in a pot. The plant shown in the illustration is 63 days from the commencement of planting outdoors in mid-June.

### DETAILED BOTANICAL DESCRIPTION

The following description of the new Chrysanthemum variety ‘Rio Dark’ is of plants grown outside, natural season, in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 50° F. at night and about 65° F. to 75° F. during the day under natural light conditions. The plants were grown outdoors in mid-June in 4 liter pots using a loamless compost. No growth retardants were used. To produce a commercial product the plants may be pinched once with the center bud removed. The typical container size for commercial growth is 4 liter (8″). It has been observed that the shelf life of the new variety is about 28 days with a response time of about 9 weeks natural season. The new variety is suitable for growth in a temperature range of 50° F. to 75° F.

The new variety may be produced as a pot plant. The following description is with respect to a plant produced as garden pot plant. In the description of this new Chrysanthemum variety, color values have been taken from The Royal Horticultural Society Colour Chart (R.H.S.C.C.).

### Plant

Plant type: Pot.

Habit: Free Branching, Round, Upright.

Height: 35-50 cm.

Width: 40-45 cm.

Branching characteristics: Free Branching, Slightly Curved.

Length of lateral branches: 15-22 cm.

Number of breaks from pinch: 7-8.

Stem color: near 152C.

Response time: 63 days natural season.

Vigor: Medium.

Shelf life: 28 days.

Disease (Susceptibility/Resistance observed): None observed to date.

Pest (Susceptibility/Resistance observed): None observed to date.

Growth retardant type and treatment: No treatment necessary The plants were grown for 9 weeks in natural season conditions.

Propagation:

Type.—Vegetative Propagation via Stem Cuttings.

Time to rooting.—12 days with soil temperatures of 70° F.

Rooting habit.—Fine and Fibrous.

### Foliage

Number of leaves per lateral branch: 7-8.

Compound or single: Single.

Arrangement of leaves: Alternate.

Shape of leaf.—Typically 5 lobed ovate.

Size of leaf.—Width (cm): 2.5-5.4 Length (cm): 5-7.

Leaf apex.—Rounded.

Base.—Cordate.

Attachment.—Petioled.

Aspect.—Slightly Undulating.

Margin.—Palmately Lobed.

Surface characteristics.—Top: Slightly Pubescent Bottom: Slightly Pubescent.

Petiole:

Color.—near 137B.

Length.—1.5-1.8 cm.

Venation: Net prominent mid-vein at underside.

Color.—upper side: near 137B underside: near 137C.

Color:

Mature leaf, upper side.—near 137A; under side: near 137C.

Young leaf, upper side.—near 137B; under side: near 137B.

### Flower

Flower appearance: Matte.

Flower type: Single.

Flower form: Flat to Slightly Cupped.

Flower shape: Round,

Flowering habit: Cyme.

Number of blossoms per branch: 18-21.

Inflorescence form: Corymibiform.

Depth of fully expanded blossoms.—2 cm.

Diameter of fully expanded blossoms.—5-6 cm.

Phyllaries:

Number.—23-25.

Color.—near 137B.

Length.—6-11 mm.

Width.—1.5-2 mm.

Texture/Appearance.—Pubescent and shiny.

Pedicel:

Length.—terminal: 12 cm; lateral: 14 cm.

Color.—near 138C.

Surface.—Pubescent.

Habit.—Straight to Slightly Curving.

Strength.—Medium.

Ray florets:

Form/Shape.—Straight.

Texture/Appearance.—Matte.

Number per flower.—115-125.

Length.—2.5-3 cm; Width: 5-6 mm.

Apex.—Rounded.

Base.—Tubular.

Margin.—Entire.

Disc florets:

Form/Shape.—Cylindrical.

Texture/Appearance.—Shiny.

Number per flower.—200-220.

Length.—0.8-1 cm; Width: 1 mm.

Diameter of disc.—1.5-1.7 cm.

Fragrance: None.

Flower bud (at onset of color):

Length.—1 cm.

Diameter.—8 mm.

Form/Shape.—Globular.

General flower color:

1. Ray florets, upper side.—Immature: near 57C Mature: near 62A Older/Fading: near 62C.

2. Ray florets, under side.—Immature: near 57C Mature: near 62A Older/Fading: near 62C.

3. Disc florets.—Immature: near 150A Mature: near 17A Older/Fading: near 17B.

4. Bud.—near 137C.

Flower progression with age: Flowers fade uniformly with age and become cupped shaped.

### Reproductive Organs

Gynoecium present on Ray and Disc florets.

Ray florets per individual flower:

Pistil number.—115-120 observed.

Stigma color.—near 144C.

Stigma shape.—Forked.

Style color.—near 6C.

Style length.—2-3 mm.

Disc florets per individual flower:

Pistil number.—180-200.

Stigma color.—near 144D.

Stigma shape.—Forked.

Style color.—near 6C.

Style length.—3-4 mm.

Androecium present on Disc florets only.

Stamen number.—50-60 observed.

Anther shape.—Tubular.

Anther color.—near 14C.

Pollen color.—near 12A.

Pollen quantity.—Fairly Abundant.

Fruit and seeds: None observed.

What is claimed is:

1. A new and distinct variety of Chrysanthemum plant, substantially as described and illustrated herein.
